(19) United States
(12) Patent Application Publication
(10) Pub. No.: US 2022/0246256 A1
(43) Pub. Date: Aug. 4, 2022

(54) USER-CUSTOMIZED ONLINE RECOMMENDATION SYSTEM AND METHOD USING HEALTH CHECKUP CHART

(71) Applicant: Hyeok HEO, Busan (KR)

(72) Inventors: Hyeok HEO, Busan (KR); Hyun Ju JEON, Busan (KR); Seok Min HEO, Busan (KR); Deung HEO, Busan (KR)

(21) Appl. No.: 17/627,682

(22) PCT Filed: Jul. 23, 2019
(86) PCT No.: PCT/KR2019/009101

§ 371 Date: Jan. 16, 2022

(30) Foreign Application Priority Data
Jul. 17, 2019 (KR) 10-2019-0086543

(51) Int. Cl. G16H 10/60; G16H 50/20; G16H 50/70; G16H 70/20; G16H 50/30

(52) U.S. Cl. CPC G16H 10/60; G16H 50/20; G16H 50/30; G16H 70/20; G16H 50/70

(57) ABSTRACT

Embodiments of the present invention may comprise: a health checkup chart image transmission step in which a user terminal photographs a user's health checkup chart and transmits the photographed health checkup chart image to a health content recommendation server; an item-specific health index extraction step in which the health content recommendation server extracts a user's health index for each item by using the health checkup chart image received from the user terminal; a user health analysis step in which the health content recommendation server extracts a health index for each item deviating from a set normal value; a deep learning-based health content information generation step in which the health content recommendation server applies the health index for each item deviating from the set normal value to deep learning of big data so as to generate deep learning-based health content information; a physical constitution analysis-based health content information generation step in which the health content recommendation server applies the health index for each item deviating from the set normal value to a physical constitution analysis tool so as to generate physical constitution analysis-based health content information; a user-customized health content information transmission step in which the health content recommendation server transmits, to the user terminal, the deep learning-based health content information and the physical constitution analysis-based health content information as user-customized health content information; a user-customized health content information recommendation step in which the user terminal displays and recommends user-customized health content information received from the health content recommendation server.

### TECHNICAL FIELD

The disclosure relates to a user-customized online recommendation system and a method thereof, and more particularly, to a customized online recommendation system which recommends a user customized health content information by using a heath checkup chart and a method thereof.

### BACKGROUND ART

An examination receiving rate of domestic health examination subject recipients is 78.2%, which is about 80% that receives health examinations, and as Korea is a leading nation in health examination worldwide with a systematic system such as a national health examination and a workplace health examination, private health examination programs are also under development changing each day based on competition between hospitals. In the industry, a market share of health examination in Korea is estimated to be greater than or equal to 4 trillion Korean won, a market share of domestic health supplements is verified at 3.8 trillion Korean won, a market share of Chinese health supplements is verified at 47 trillion Korean won, and a market share of U.S. health supplements is verified at 40.1 trillion Korean won.

Now, health related products are changing from a ‘comprehensive type’ to a specific ‘customized type’ following a trend of ‘self-medication’ of looking after one's own health.

Customized health information providing services of the related art have provided general health information, disease information, or the like through an internet portal or health information websites. In addition, it is typical to follow a questionnaire type such as searching a standardized dictionary type or searching by each symptom which are provided by a service provider for a user to verify one's state of health.

Technologies of the related art as described above have mainly been platforms that derive a result by analyzing the state of health of the user through surveying daily habits, dietary habits, and the like when checking the state of health of the user and recommend necessary elements or exercise that benefit one's health according to a result value of the state of health of the user, but the state of health of the user according to the survey result is not reliable official data, and there is the problem of inconvenience occurring due to having to obtain information through much surveying in order to check the state of health of the user.

### DISCLOSURE

### Technical Problem

The technical problem of the disclosure lies in checking one's state of health in real time more accurately from a preventive medicine aspect of an individual health state by analyzing the state of health of the user and matching elements necessary to items deviating from a normal value when the user photographs a health checkup chart with a user terminal such as a smartphone and uploads the photographed image by executing a corresponding app (or, application), and recommending health (supplementary) foods and health teas, health drinks, other health enhancement products and services, and the like, which are most optimized to individual constitution by applying big data and deep learning technology to the matched result value.

### Technical Solution

According to an embodiment, a user-customized online recommendation method using a health checkup chart includes a health checkup chart image transmission step of photographing, by a user terminal, a health checkup chart of a user and transmitting the photographed health checkup chart image to a health content recommendation server; an item-specific health index extraction step of using, by the health content recommendation server, the health checkup chart image received from the user terminal to extract an item-specific health index of the user; a user health analysis step of extracting, by the health content recommendation server, the item-specific health index which deviated a set normal value; a deep learning-based health content information generation step of generating, by the health content recommendation server, deep learning-based health content information by applying the item-specific health index which deviated a set normal value to a deep learning training of big data; a physical constitution analysis-based health content information generation step of generating, by the health content recommendation server, the physical constitution analysis-based health content information by applying a year, month, day, and time of a user's birth to a physical constitution analysis tool; and a user-customized health content information transmission step of transmitting, by the health content recommendation server, the deep learning-based health content information and physical constitution analysis-based health content information to the user terminal as user-customized health content information; and a user-customized health content information recommendation step of displaying recommending, by the user terminal, user-customized health content information received from the health content recommendation server.

The item-specific health index extraction step may include a step of extracting text from the health checkup chart image received from the user terminal; and a step of extracting an item-specific health index of a user from the extracted text.

The physical constitution analysis-based health content information generation step is characterized in that the physical constitution analysis-based health content information may be generated by applying to the physical constitution analysis tool which is based on a theory of a Chinese medical text, Study on Ounyukgi.

The physical constitution analysis-based health content information generation step may include a year, month, day, and time of a user's birth input step of extracting a data of birth from the health checkup chart image and receiving a time of birth from the user to receive a ‘year, month, day, and time of a user's birth’ which is the date of birth and time of birth of the user; a user Saju Palja conversion step of applying the ‘year, month, day, and time of the user's birth’ to a thousand-year calendar to convert a ‘user Saju Palja’; a user five element conversion step of converting the ‘user Saju Palja’ to ‘user five elements’ which is in a five element form of wood, fire, earth, metal, and water based on a five element theory, the principle of which interprets a whole of natural elements or a human related phenomenon; a five element-based health index identification step of identifying a ‘five element-based health index’ which is a user health state that is based on the ‘user five elements’; and a five element-based health content information provision step of generating the physical constitution analysis-based health content information which is based on the ‘five element-based health index.’

The five element-based health content information provision step may include a temporary weight value health index calculation step of calculating a ‘temporary weight value health index’ by adding a pre-set temporary weight value to the ‘five element-based health index’ for each of the five elements; a similarity identification step of comparing the calculated temporary weight value health index for each of the five elements with a user health checkup chart health index to identify whether it is within a health state similarity range of an error range; a matching weight value determination step of determining a temporary weight value as a matching weight value based on the temporary weight value health index for each of the five elements and the user health checkup chart health index being within the health state similarity range, and determining a matching weight value while changing the temporary weight value until the temporary weight value health index for each of the five elements and the user health checkup chart health index fall within the health state similarity range based on the temporary weight value health index for each of the five elements and the user health checkup chart health index deviating from the health state similarity range; a weight value record step of recording the determined matching weight value for each of the five elements to a weight value determination DB; a basic weight value determination step of determining an average value of matching weight values for each of the five elements that is recorded as a basic weight value based on a recoded number of matching weight values for each of the five elements recorded in the weight value determination DB exceeding a set threshold value; and a basic weight value applied health content information provision step of applying the basic weight value to the user five elements according to the user Saju Palja that is based on the ‘year, month, day, and time of the user's birth’ to calculate a basic weight value health index, and providing health content information that is based on the calculated basic weight value health index.

The user-customized health content information may include at least one of user-customized tea information, user-customized drink information, user-customized medicinal herb information, user-customized food information, user-customized health enhancement product information, user-customized clinic information, user-customized physician information, and user-customized exercise information.

When the user-customized health content information is the user-customized tea information, the user-customized drink information, the user-customized medicinal herb information, or the user-customized food information, the user-customized health content information recommendation step may include extracting only information which matches with the physical constitution analysis-based health content information from among the deep learning-based health content information generated through the deep learning-based health content information generation step to display as the user-customized health content information.

The user-customized health content information recommendation step may include a user preference analysis step of analyzing a user preference on a treatment remedy; and a user preference based display step of displaying only user-customized health content information corresponding to the analyzed user preference on the treatment remedy from among the deep learning-based health content information, and the physical constitution analysis-based health content information.

The user preference analysis step may include receiving a survey response from the user on which treatment method from among a folk remedy and a medical treatment remedy is preferred and identifying the user preference on the treatment remedy based on the input survey response, and the user preference-based display step may include displaying only the user health content information of any one from among the deep learning-based health content information and the physical constitution analysis-based health content information according to the identified user preference on the treatment remedy.

The user preference-based display step may include displaying only the deep learning-based health content information based on the user preference on the treatment remedy being identified as a medical treatment remedy, and displaying only the physical constitution analysis-based health content information based on the user preference on the treatment remedy being identified as a folk remedy.

According to an embodiment, a user-customized online recommendation system includes a user terminal configured to photograph a health checkup chart of a user and transmit the photographed health checkup chart image to a health content recommendation server, and display and recommend the user-customized health content information received from the health content recommendation server; a health content recommendation server configured to extract an item-specific health index of the user by using the health checkup chart image received from the user terminal, generate deep learning-based health content information by applying the item-specific health index to a deep learning training of big data, and transmit the deep learning-based health content information and the physical constitution analysis-based health content information to the user terminal as user-customized health content information after generating the physical constitution analysis-based health content information by applying the item-specific health index to a physical constitution analysis tool; and a wired/wireless communication network configured to provide a wired communication or a wireless communication between the user terminal and the health content recommendation server.

### Advantageous Effects

According to an embodiment of the disclosure, by recommending an optimum health enhancement product and service, and the like through big data, a deep learning technology, a constitution analysis, and the like with respect to a health checkup chart provided by each nation, contribution may be made to national health and strengthening of health of cosmopolitans.

In addition, according to an embodiment, by recommending optimum health related products and information, and the like to users, it may lead to import substitution and export increase by providing optimum products and services to not only Korea but also to Asian countries such as China, Japan, and the like, and positioning as a leading service in world renowned preventive medicine may be expected.

In addition, according to an embodiment of the disclosure, accumulated data associated with the health of the user which is secured continuously through the health check up chart, and the like according to a voluntary decision of the user may be utilized as health big data which is accumulated hereafter.

### DESCRIPTION OF DRAWINGS

FIG. 1 is a configuration view of a user-customized online recommendation system which uses a health checkup chart according to an embodiment of the disclosure;

FIG. 2 is a view illustrating an implementation of an operation of a user-customized online recommendation system according to an embodiment of the disclosure;

FIG. 3 is a flowchart of a user-customized online recommendation system which uses a health checkup chart according to an embodiment of the disclosure;

FIG. 4 is an example view of a health checkup chart according to an embodiment of the disclosure;

FIG. 5 is a view illustrating a generating of deep learning-based health content information through deep learning according to an embodiment of the disclosure;

FIG. 6 is a view illustrating a generating of deep learning-based health content information using a constitution analysis tool according to an embodiment of the disclosure;

FIG. 7 is a flowchart illustrating a physical constitution analysis-based health content information generation step according to an embodiment of the disclosure;

FIG. 8 is an example view of placing weight value for each of five elements according to an embodiment of the disclosure;

FIG. 9 is a view illustrating compatibility and incompatibility of the five elements;

FIG. 10 is a view illustrating a recommending of a user-customized health content information through a primary analysis and deep learning of a health checkup chart and a secondary analysis on a study of Ounyukgi according to an embodiment of the disclosure;

FIG. 11 is an example view of extracting only information which matches with a physical constitution analysis-based health content information from among a deep learning-based health content information according to an embodiment of the disclosure; and

FIG. 12 is an example view of providing only health content information according to a user preference on a treatment remedy according to an embodiment of the disclosure.

### MODE FOR INVENTION

Hereinbelow, advantages and features of the disclosure, and a method of achieving the same will be made clear with reference to embodiments which are described in detail below together with the accompanying drawings. However, the disclosure is not limited to the embodiments described below and may be implemented to various forms different from one another, and as the embodiments are provided to fully convey a scope of the disclosure to those of ordinary skill in the art to which the disclosure pertains, the disclosure may be defined by the scope of the claims. In addition, in case it is determined that in describing the disclosure, a detailed description of related known technologies may unnecessarily confuse the gist of the disclosure, the detailed description will be omitted.

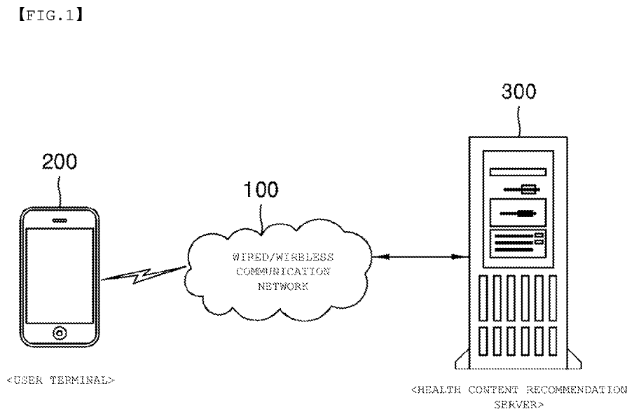

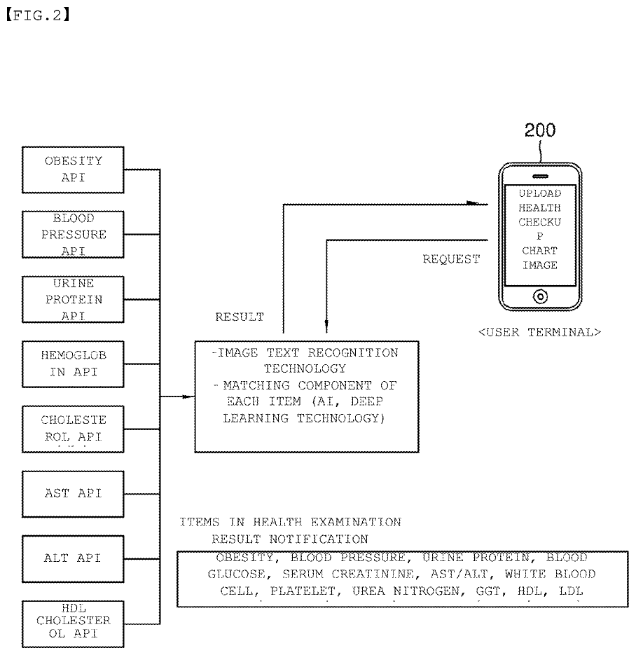

FIG. 1 is a configuration view of a user-customized online recommendation system which uses a health checkup chart according to an embodiment of the disclosure, and FIG. 2 is a view illustrating an implementation of an operation of a user-customized online recommendation system according to an embodiment of the disclosure.

The disclosure describes classifying in API for each item such as a degree of obesity, an eye sight, a blood pressure level, and a diabetic level which are in the health checkup chart, documenting the same when the user uploads an image of a result notification by mobile or web, recognizing a value deviating from a normal value for each item, and recommending a customized health enhancement product necessary to the user by analyzing what is previously classified in API for each item with big data such as medicine, pharmacology, Oriental medicine, and study of food and nutrition, utilizing deep learning technology to find an optimum pharmacological element suitable to an individual and matching the corresponding health enhancement product.

To this end, a user customized online recommendation system of the disclosure may include a wired/wireless communication network 100, a user terminal 200, and a health content recommendation server 300 as shown in FIG. 1.

The wired/wireless communication network 100 may provide a wired communication or a wireless communication between the user terminal 200 and the health content recommendation server 300. When the wired/wireless communication network 100 is implemented with a wireless communication network, data communication may be carried out by using a wireless mobile communication network comprised of a base transceiver station (BTS), a mobile switching center (MSC), and a home location register (HLR). In addition, when the wired/wireless communication network 100 is implemented with a wired communication network, it may be implemented through a network communication network but data communication may be carried out according to internet protocols such as a transmission control protocol/internet protocol (TCP/IP).

The user terminal 200 may be a terminal used by the user, and although a smartphone is illustrated in the drawings, various terminals such as a tablet, a desktop PC, and a notebook may correspond thereto.

The user terminal 200 may photograph the health checkup chart of the user and transmit the photographed health checkup chart image to the health content recommendation server 300. Here, the health checkup chart may be a health checkup notification which is carried out every two years by the Health Insurance Corporation, a comprehensive health checkup result report which the user pays with user's money and performs, and the like.

The user terminal 200 may display and recommend user-customized health content information received from the health content recommendation server 300. Here, the user-customized health content information may correspond to deep learning-based health content information in which an item-specific health index of the health checkup chart is applied to the deep learning training of big data, physical constitution analysis-based health content information which is applied to a physical constitution analysis tool, and the like.

Accordingly, as illustrated in FIG. 2, the user may check one's state of health more accurately in real-time from a preventive medicinal aspect of the user's state of health by analyzing the user's state of health and matching elements necessary to items deviating from the normal value when the health checkup chart (health checkup result notification) is photographed with the user terminal 200 and the photographed image is uploaded by executing a corresponding app, and recommending health (supplementary) foods and health teas, health drinks, health enhancement products, and the like, which are most optimized to an individual's physical constitution by applying big data and deep learning technology to the matched result value.

The health content recommendation server 300 may include, in terms of hardware, the same configuration as a web server of the related art, and in terms of software, a program module which performs several functions implemented through languages of various forms such as C, C++, Java, Visual Basic, and Visual C. In addition, it may be implemented using a web server program which is provided variously according to an operating system such as dos, Window, Linux, Unix, and Macintosh in a typical hardware for a server.

The health content recommendation server 300 may use the health checkup chart image which is received from the user terminal 200 to extract the item-specific health index which includes the degree of obesity (body mass index), the eye sight, the blood pressure level, and the diabetic level of the user. Further, deep learning-based health content information is generated by applying the item-specific health index deviating from a pre-set normal value to the deep learning training of big data, and the physical constitution analysis-based health content information is generated by applying the item-specific health index deviating from a set normal value to the physical constitution analysis tool. The generated deep learning-based health content information and the physical constitution analysis-based health content information may be transmitted to the user terminal 200 as user-customized health content information. The above will be described below with FIGS. 2 to 12.

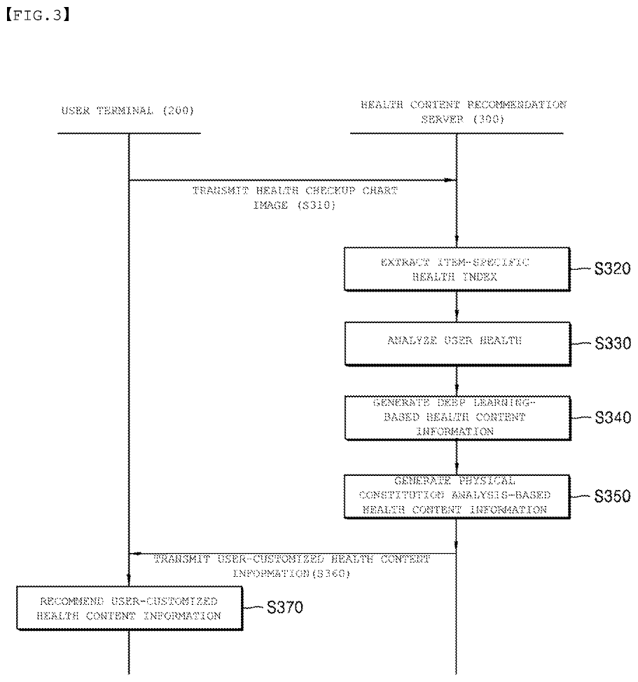

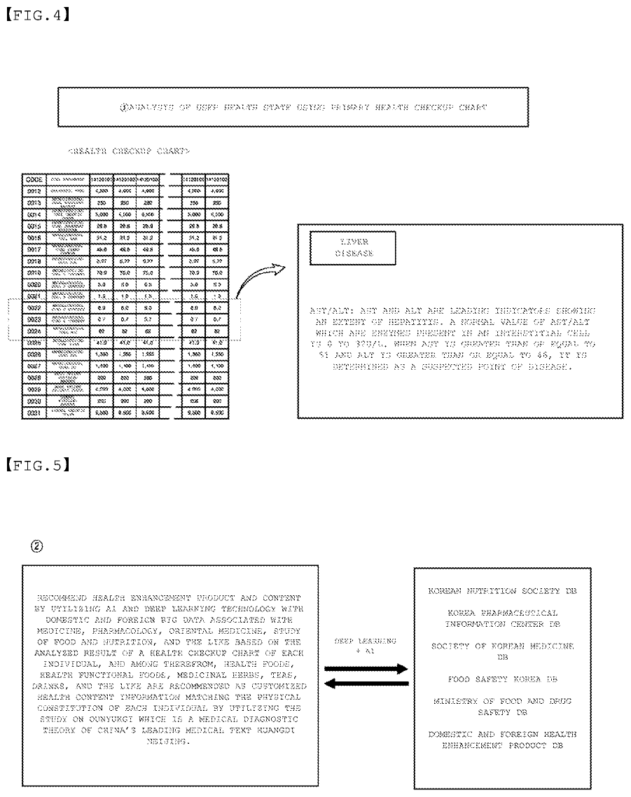

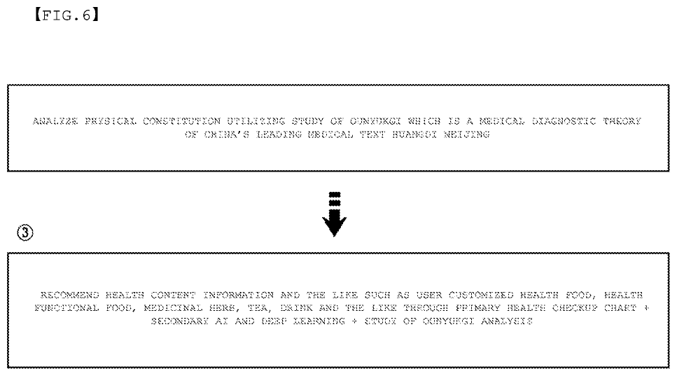

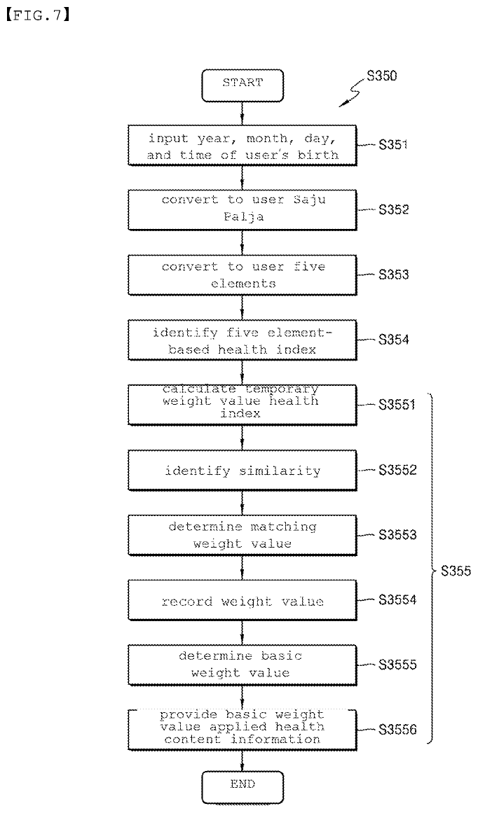

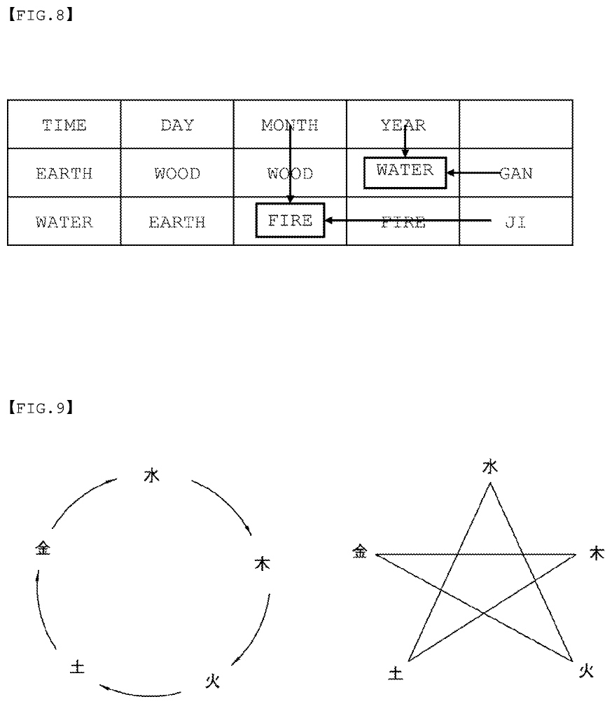

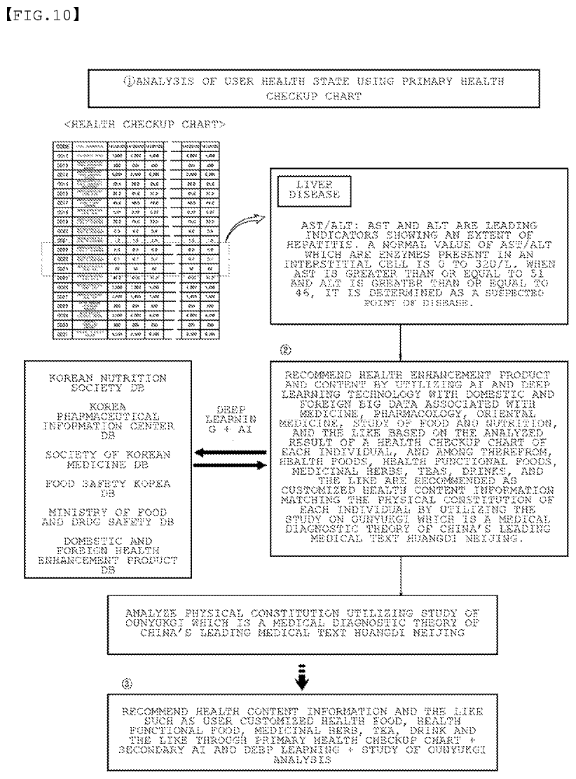

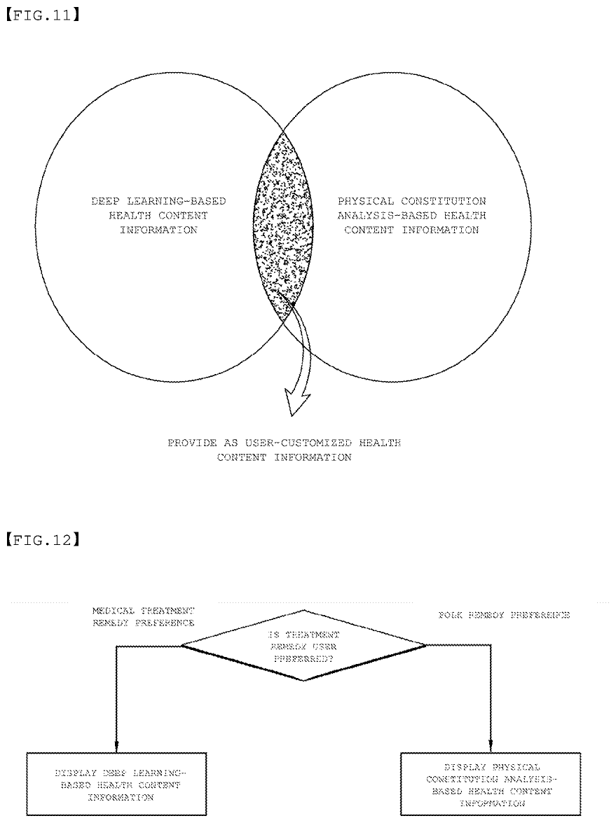

FIG. 3 is a flowchart of a user-customized online recommendation method which uses a health checkup chart according to an embodiment of the disclosure, FIG. 4 is an example view of a health checkup chart according to an embodiment of the disclosure, FIG. 5 is a view illustrating a generating of deep learning-based health content information through deep learning training according to an embodiment of the disclosure, FIG. 6 is a view illustrating a generating of deep learning-based health content information using a physical constitution analysis tool according to an embodiment of the disclosure, FIG. 7 is a flowchart illustrating a physical constitution analysis-based health content information generation step according to an embodiment of the disclosure, FIG. 8 is an example view of placing weight value for each of five elements according to an embodiment of the disclosure, FIG. 9 is a view illustrating compatibility and incompatibility of the five elements, FIG. 10 is a view illustrating a recommending of a user-customized health content information through a primary analysis and deep learning of a health checkup chart and a secondary analysis of a study on Ounyukgi [symbol] according to an embodiment of the disclosure, FIG. 11 is an example view of extracting only information which matches with a physical constitution analysis-based health content information from among a deep learning-based health content information according to an embodiment of the disclosure, and FIG. 12 is an example view of providing only health content information according to a user preference on a treatment remedy according to an embodiment of the disclosure.

The user-customized online recommendation method of the disclosure may include a health checkup chart image transmission step (S310) of the user's health checkup chart being photographed by the user terminal 200 and the photographed health checkup chart image being transmitted to the health content recommendation server 300, an item-specific health index extraction step (S320) of the item-specific health index which includes the degree of obesity (body mass index), the eye sight, the blood pressure level, and the diabetic level of the user being extracted by the health content recommendation server 300 using the health checkup chart image received from the user terminal 200, a user health analysis step (S330), a deep learning-based health content information generation step (S340) of the deep learning-based health content information being generated by health content recommendation server 300 and the item-specific health index being applied to the deep learning training of big data, physical constitution analysis-based health content information generation step (S350) of the physical constitution analysis-based health content information being generated by the health content recommendation server 300 applying a year, month, day, and time of the user's birth to the physical constitution analysis tool, a user-customized health content information transmission step (S360) of the deep learning-based health content information and the physical constitution analysis-based health content information being transmitted by the health content recommendation server 300 to the user terminal 200 as user-customized health content information, and a user-customized health content information recommendation step (S370) of the user-customized health content information received from the health content recommendation server 300 being displayed and recommended by the user terminal 200, as illustrated in FIG. 3. The above will be described below.

The health checkup chart image transmission step (S310) may be a step in which the user's health checkup chart is photographed by the user terminal 200 and the photographed health checkup chart image is transmitted to the health content recommendation server 300.

As a reference, the health checkup chart may be a notification on a health checkup result which is carried out by the National Insurance Corporation and the state of health of the user that received the health checkup may be checked. For example, as illustrated in FIG. 4, AST and ALT are leading indicators showing an extent of hepatitis. A normal level for AST and ALT which are enzymes contained within hepatocytes is 0˜32 U/L. Based on AST being greater than or equal to 51, and ALT being greater than or equal to 46, the user may be determined as suspect to disease.

The item-specific health index extraction step (S320) may be a step in which the health checkup chart image received from the user terminal 200 is used by the health content recommendation server 300 to extract the item-specific health index which includes the degree of obesity (body mass index), the eye sight, the blood pressure level, and the diabetic level of the user.

The item-specific health index extraction step (S320) as described above may first include the step of extracting a text from the health checkup chart image received from the user terminal 200. For example, the text may be extracted by deciphering the health checkup chart image with an optical character recognition (OCR).

After the text is extracted, the step of extracting the item-specific health index may be included. Here, the item-specific health index may include the degree of obesity (body mass index), the eye sight, the blood pressure level, and the diabetic level of the user, and other various health indexes may be included in addition thereto.

The user health analysis step (S330) may be a step in which the item-specific health index deviating from the set normal value is extracted by the health content recommendation server 300. For example, it is a step of identifying whether there is an item-specific health index deviating from the pre-set normal value such as whether there is deviation from a normal high blood pressure level, and whether there is deviation from a reference value in the degree of obesity.

The deep learning-based health content information generation step (S340) may be a step in which the item-specific health index is applied to the deep learning training of big data by the health content recommendation server 300 to generate the deep learning-based health content information as illustrated in FIG. 5. That is, the user health content information matching the user's item-specific health index may be generated as deep learning-based health content information based on the deep learning trained result using a DB of the Korean Nutrition Society, a DB of the Korea Pharmaceutical Information Center, a DB of the Society of Korean Medicine, a DB of Food Safety Korea, a DB of the Ministry of Food and Drug Safety, and other DBs associated with health enhancement products domestically and in foreign nations. For example, in the case of a user with high blood pressure levels, user health content information which may improve the corresponding blood pressure levels to be lower may be generated by extracting through the deep learning training result. As a reference, the deep learning training (deep learning) may be a technology used to group or classify objects or data as known in the art, and may be a technology for inputting much data to a computer and classifying those similar with one another. It is a machine learning method which was proposed to overcome limitations of an artificial neural network.

Here, the deep learning-based health content information may be included with at least one of user-customized tea information, user-customized drink information, user-customized medicinal herb information, user-customized food information, user-customized health supplement information, user-customized clinic information, user-customized physician information, user-customized exercise information, and the like, and other various information may be included in addition thereto.

Furthermore, when providing the user-customized tea information or the user-customized drink information, the medicinal herbs of Oriental medicine and the components of Western medicine may be respectively shown first, and Oriental medicine and Western medicine including the corresponding components may be suggested when selected by the user. For example, when strengthening of the stomach is necessary, the medicinal herb ‘Pinellia ternata’ may be shown for Oriental medicine, and component ‘catechin’ may be shown for Western medicine, ‘Banha Sasim-Tang’ or ‘Cabagin’ may be shown when selected by the user.

The physical constitution analysis-based health content information generation step (S350) may be a step in which the year, month, day, and time of the user's birth is applied to the physical constitution analysis tool by the health content recommendation server 300 to generate the physical constitution analysis-based health content information as illustrated in FIG. 6.

Likewise, the physical constitution analysis-based health content information may be included with at least one of the user-customized tea information, the user-customized drink information, the user-customized medicinal herb information, the user-customized food information, the user-customized health supplement information, the user-customized clinic information, the user-customized physician information, the user-customized exercise information, and the like, and other various information may be included in addition thereto.

Here, the physical constitution analysis tool may be used as an analysis tool of the physical constitution which is an analysis tool based on a theory of the study on Ounyukgi which is a diagnostic medicine theory in a Chinese medical text “Huangdi Neijing”. That is, the physical constitution analysis-based health content information generation step may generate the physical constitution analysis-based health content information by applying to the physical constitution analysis tool which is based on the Chinese medical text study on Ounyukgi theory, and will be described below with FIGS. 7 to 9.

Referring to FIG. 7, the physical constitution analysis-based health content information generation step (S350) may include a year, month, day, and time of a user's birth input step (S351), a user Saju Palja (Four Pillars of Destiny) conversion step (S352), a user five element conversion step (S353), a five element-based health index identification step (S354), and a five element-based health content information provision step (S355).

The year, month, day, and time of the user's birth input step (S351) may be a step in which the ‘year, month, day, and time of the user's birth’ which is the date of birth and time of birth of the user is received. The input may include extracting the date of birth from the health checkup chart image and receiving the time of birth from the user.

The user Saju Palja conversion step (S352) may be a step in which the ‘year, month, day, and time of the user's birth’ is applied to a thousand-year calendar to convert the ‘user Saju Palja.’ As a reference, the thousand-year calendar describes a Taese of each year, a Wolgun and a Daeso of each month, an Iljin of each day, a Sak⋅Hyun⋅Mang of a month, an Ipkiilsi of a 24 Solar Term, an Ilwol 5-haeng, that is the daily location of chiljung, the location of 4-yeo every 10 days, and the like.

Accordingly, if the time is input to the date of birth as shown below, the Saju Palja according to the thousand-year calendar may be calculated as shown in [Table 1].

TABLE 1

| Time | Day | Month | Year | |
|---|---|---|---|---|
| [symbol] | [symbol] | [symbol] | [symbol] | Gan |
| [symbol] | [symbol] | [symbol] | [symbol] | Ji |

The user five element conversion step (S353) may be a step in which the ‘user Saju Palja’ is converted to ‘user five elements’ which is in the five element form of wood, fire, earth, metal and water based on a five element theory, the principle of which interprets a whole of the natural elements or a human related phenomenon (including forms of matter or even spirit).

The Saju Palja may be converted to wood⋅fire⋅earth⋅metal water which are the five elements, and for example, based on having a Saju Palja as shown in [Table 1] above, it may be converted to the five element of [Table 2] below.

TABLE 2

| Time | Day | Month | Year | |
|---|---|---|---|---|
| earth | wood | wood | water | Gan |
| water | earth | fire | fire | Ji |

Accordingly, in the case of a user who has the five element of [Table 2] above, the user may have earth 2, wood 2, water 2, and fire 2. As a reference, in the five element theory, the metal may mean a lung function, the wood may mean a liver function, the earth may mean a stomach function, the water may mean a kidney function, and the fire may mean a heart function.

The five element-based health index identification step (S354) may be a step based on the ‘user five elements’ for identifying the ‘five element-based health index’ which is the user's health state. For example, the user with the five elements of [Table 2] above may be interpreted as having a weak lung function because there is no metal and the remaining functions being in good condition.

The five element-based health content information provision step (S355) may b a step for generating the physical constitution analysis-based health content information which is based on the ‘five element-based health index.’

The five element-based health content information provision step (S355) may be provided in two methods as described below.

The first method describes of providing health content information based on the five element-based health index itself. For example, because the user with the five elements of [Table 2] above is interpreted as having a weak lung function as there is no metal and the remaining functions being in good condition, health content information which may improve the lung function may be provided.

The second method describes of providing health content information by using a weight value. For example, weight values may be placed for each of the five elements, and for example, ‘fire’ which is ‘Wol-Ji’ may set +1 as the weight value, and ‘water’ which is ‘Yeon-Gan’ may set ½ as the weight value. When the weight values are placed, the user with the five elements of [Table 2] may be changed to fire 3, water 1, wood 2, and fire 2, and thereby interpreted as having very good heart function, normal kidney function, weak lung function, and the remaining in good condition.

The result described above may be compared with the health checkup chart to determine whether it is a match, and weight values are stored if a strong part and a weak part are a match (i.e., if a match occurs by applying ‘Wol-Ji’ +1, the weight value may be stored). In case a match does not occur, the weight value may be differently adjusted. The above may be continuously accumulated in a database. As described above, based on the weight value for each of the five elements being fixed by applying targeting a large number of people, the health content information may be generated by using the fixed weight value. That is, with just the ‘year, month, day, and time of the user's birth,’ weight value may be applied to the five element of the user to provide the user's health content information matching thereto.

Describing the second method which provides health content information by using the weight value (S355) in greater detail, a temporary weight value health index calculation step (S3551), a similarity identification step (S3552), a matching weight value determination step (S3553), a weight value record step (S3554), a basic weight value determination step (S3555), and a basic weight value applied health content information provision step (S3556) may be included.

The temporary weight value health index calculation step (S3551) may be a step in which the ‘temporary weight value health index’ is calculated by adding a pre-set temporary weight value to the ‘five element-based health index’ for each of the five elements. The temporary weight value may be a weight value which is allocated initially, and for example, may correspond to 1, 0.5, and the like for each of the five elements.

The similarity identification step (S3552) may be a step in which the calculated temporary weight value health index for each of the five elements is compared with the user health checkup chart health index to identify whether it is within the health state similarity range within an error range. For example, based on ‘fire’ which is ‘Wol-Ji’ applying +1 as the temporary weight value and ‘water’ which is ‘Yeon-Gan’ applying ½ as the temporary weight value, the user with the five elements of [Table 2] may be changed to fire 3, water 1, wood 2, and fire 2, which is interpreted to the heart function being in very good condition, the kidney function being normal, the lung function being weak, and the remaining being good, and if the health index of the lung in the health checkup chart corresponds to a bad index, the health state of the lung may be determined as within the similarity range as with the temporary weight value health index for each of the five elements, and alternatively, if the health index of the lung in the health checkup chart corresponds to a good index, the health state of the lung is determined as deviating from the similarity range with the temporary weight value health index for each of the five elements.

The matching weight value determination step (S3553) may be a step of determining the temporary weight value as the matching weight value when the temporary weight value health index for each of the five elements and the user health checkup chart health index are within the health state similarity range, and when the temporary weight value health index for each of the five elements and the user health checkup chart health index are deviated from the health state similarity range, determining the matching weight value while changing the temporary weight value until the temporary weight value health index for each of the five elements and the user health checkup chart health index fall within the health state similarity range. For example, based on the health state of the lungs being within the similarity range with the temporary weight value health index for each of the five elements if the health index of the lungs corresponds to a bad index in the health checkup chart, ‘fire’ which is ‘Wol-Ji’ may be determined with the temporary weight value of +1 as the matching weight value, and alternatively, based on the health state of the lungs being determined as deviated from the similarity range with the temporary weight value health index for each of the five elements because the health index of the lungs corresponds to a good index in the health checkup chart, the matching weight value is finally determined by continuously changing the temporary weight value to 0.5. 2, 3, and the like, until reaching the similarity range. As a reference, referring to FIG. 9, a compatibility and an incompatibility table of the five elements are shown, and the weight value may be additionally adjusted by +1 if there is a line based sidewards which is compatible, ½ if there is a line which is incompatible, and the like. For example, if ‘Si-Ji’ is wood, and ‘Il-Ji’ is ‘fire,’ +1 may be applied to ‘fire.’

The weight value record step (S3554) may be a step in which the determined matching weight value for each of the five elements is recorded in a weight value determination DB.

The basic weight value determination step (S3555) may be a step in which an average value of the recorded matching weight value for each of the five elements is determined as the basic weight value when a recorded number of the matching weight value for each of the five elements recorded in the weight value determination DB exceeds a set threshold value. For example, based on the recorded number of the matching weight value to ‘fire’ which is ‘Wol-Ji’ being more than 20 times, the average value which adds all of the matching values recorded for 20 times and divides the total by 20 is determined as the basic weight value.

The basic weight value applied health content information provision step (S3556) may be a step in which the basic weight value health index is calculated by applying the basic weight value to the user five elements according to the user Saju Palja which is based on the ‘year, month, day, and time of the user's birth’ to provide health content information that is based on the calculated basic weight value health index.

For example, based on the recorded number of matching weight values to ‘fire’ which is ‘Wol-Ji’ being more than 20 times, and based on the basic weight value which is the average value that adds all of the matching weight values recorded for 20 times and divides the total by 20 being determined as 0.8, when user A has 3 ‘fire’ which is ‘Wol-Ji,’ the basic weight value health index calculated is 2.4, which is calculated as 3×0.8. Accordingly, ‘fire’ of the five elements corresponding to the heart function is determined to the health index of 2.4, and user A may be provided with health content information corresponding thereto.

Meanwhile, the user-customized health content information transmission step (S360) may be a step in which the health content recommendation server 300 transmits the deep learning-based health content information and the physical constitution analysis-based health content information to the user terminal 200 as user-customized health content information.

The user-customized health content information recommendation step (S370) may be a step in which the user terminal 200 displays and recommends the user-customized health content information received from the health content recommendation server 300. Accordingly, as illustrated in FIG. 10, the user-customized health content information may be recommended through the primary analysis and deep learning of the health checkup chart and the secondary analysis of the study on Ounyukgi.

Meanwhile, based on the user-customized health content information being the user-customized tea information, the user-customized drink information, the user-customized medicinal herb information, or the user-customized food information, the deep learning-based health content information through deep learning training and the physical constitution analysis-based health content information through the physical constitution analysis tool may match each other, or may have different values from each other. Accordingly, there is a need for recommending and providing the information to the user based on a pre-set standard to prevent user confusion.

Recommendation provision may be carried out in the two methods described below.

The first method may include extracting, based on the user-customized health content information being the user-customized tea information, the user-customized drink information, the user-customized medicinal herb information, or the user-customized food information, only information that matches with the physical constitution analysis-based health content information from among the deep learning-based health content information generated through the deep learning-based health content information generation step (S340) by the user-customized health content information recommendation step (S370) as illustrated in FIG. 11 to display as the user-customized health content information.

Accordingly, by providing to the user only information corresponding to an intersection of the deep learning-based health content information and the physical constitution analysis-based health content information, reliability of the recommendation may be raised so that incompatible food recommendations and the like are not made.

A different second method may include a user preference analysis step of analyzing the user preference on the treatment remedy, and a user preference-based display step of displaying only the user-customized health content information which corresponds to the user preference of the analyzed treatment remedy from among the primary analyzed deep learning-based health content information and the secondary analyzed physical constitution analysis-based health content information.

Here, the user preference analysis step may include receiving a response to a survey from the user on which treatment remedy is preferred from among a folk remedy and a medical treatment remedy and identify the user preference on the treatment remedy based on the input survey response. For example, the user preference may be identified by having the user fill in survey questions indicating whether objective scientific facts are preferred or whether home remedies are preferred.

When the analysis on the user preference is carried out, the user preference-based display step may include displaying only the user health content information of any one from among the deep learning-based health content information, or the secondary analyzed physical constitution analysis-based health content information according to the user preference on the identified treatment remedy. That is, as illustrated in FIG. 12, based on the user preference on the treatment remedy being identified as medical treatment remedy, only the deep learning-based health content information may be displayed, and based on the user preference on the treatment remedy being identified as folk remedy, only the physical constitution analysis-based health content information may be displayed.

Accordingly, based on the user preference preferring objective scientific facts, the deep learning-based health content information may be provided, and alternatively, based on the user preference preferring Oriental medicine or folk remedies, the physical constitution analysis-based health content information is provided satisfying the needs of all users.

The embodiments in the description of the disclosure described above have been selected and provided as best mode examples to assist in the understanding of those skilled in the art from among the various practicable examples, and the technical idea of the disclosure it not necessarily limited to the specific embodiments disclosed herein and various changes and modifications, equivalents and/or alternatives of the embodiments may be made without departing from the scope and spirit of the disclosure.

### MODE FOR INVENTION

The mode for the practice of the invention has been described with the Best Mode for practicing the invention above.

### INDUSTRIAL APPLICABILITY

The disclosure relates to a user-customized online recommendation system and method using a health checkup chart, and because it is implemented by computer technology to be performed on a computer (user terminal, health content recommendation server), the invention has industrial applicability.

1. A user-customized online recommendation method using a health checkup chart, the method comprising:
a health checkup chart image transmission step of photographing, by a user terminal, a health checkup chart of a user and transmitting the photographed health checkup chart image to a health content recommendation server;
an item-specific health index extraction step of using, by the health content recommendation server, the health checkup chart image received from the user terminal to extract an item-specific health index of the user;
a user health analysis step of extracting, by the health content recommendation server, the item-specific health index which deviated a set normal value;
a deep learning-based health content information generation step of generating, by the health content recommendation server, deep learning-based health content information by applying the item-specific health index which deviated a set normal value to a deep learning training of big data;
a physical constitution analysis-based health content information generation step of generating, by the health content recommendation server, the physical constitution analysis-based health content information by applying a year, month, day, and time of a user's birth to a physical constitution analysis tool; and
a user-customized health content information transmission step of transmitting, by the health content recommendation server, the deep learning-based health content information and physical constitution analysis-based health content information to the user terminal as user-customized health content information; and a user-customized health content information recommendation step of displaying recommending, by the user terminal, user-customized health content information received from the health content recommendation server.

2. The method of claim 1, wherein the item-specific health index extraction step comprises:
a step of extracting text from the health checkup chart image received from the user terminal; and
a step of extracting an item-specific health index of a user from the extracted text.

3. The method of claim 1, wherein the physical constitution analysis-based health content information generation step is characterized in that the physical constitution analysis-based health content information is generated by applying to the physical constitution analysis tool which is based on a theory of a Chinese medical text, Study on Ounyukgi.

4. The method of claim 3, wherein the physical constitution analysis-based health content information generation step comprises:
a year, month, day, and time of a user's birth input step of extracting a data of birth from the health checkup chart image and receiving a time of birth from the user to receive a ‘year, month, day, and time of a user's birth’ which is the date of birth and time of birth of the user;
a user Saju Palja conversion step of applying the ‘year, month, day, and time of the user's birth’ to a thousand-year calendar to convert a ‘user Saju Palja’;
a user five element conversion step of converting the ‘user Saju Palja’ to ‘user five elements’ which is in a five element form of wood, fire, earth, metal, and water based on a five element theory, the principle of which interprets a whole of natural elements or a human related phenomenon;
a five element-based health index identification step of identifying a ‘five element-based health index’ which is a user health state that is based on the ‘user five elements’; and
a five element-based health content information provision step of generating the physical constitution analysis-based health content information which is based on the ‘five element-based health index.’

5. The method of claim 4, wherein the five element-based health content information provision step comprises:
a temporary weight value health index calculation step of calculating a ‘temporary weight value health index’ by adding a pre-set temporary weight value to the ‘five element-based health index’ for each of the five elements;
a similarity identification step of comparing the calculated temporary weight value health index for each of the five elements with a user health checkup chart health index to identify whether it is within a health state similarity range of an error range;
a matching weight value determination step of determining a temporary weight value as a matching weight value based on the temporary weight value health index for each of the five elements and the user health checkup chart health index being within the health state similarity range, and determining a matching weight value while changing the temporary weight value until the temporary weight value health index for each of the five elements and the user health checkup chart health index fall within the health state similarity range based on the temporary weight value health index for each of the five elements and the user health checkup chart health index deviating from the health state similarity range;
a weight value record step of recording the determined matching weight value for each of the five elements to a weight value determination DB;
a basic weight value determination step of determining an average value of matching weight values for each of the five elements that is recorded as a basic weight value based on a recoded number of matching weight values for each of the five elements recorded in the weight value determination DB exceeding a set threshold value; and
a basic weight value applied health content information provision step of applying the basic weight value to the user five elements according to the user Saju Palja that is based on the ‘year, month, day, and time of the user's birth’ to calculate a basic weight value health index, and providing health content information that is based on the calculated basic weight value health index.

6. The method of claim 1, wherein the user-customized health content information comprises at least one of user-customized tea information, user-customized drink information, user-customized medicinal herb information, user-customized food information, user-customized health enhancement product information, user-customized clinic information, user-customized physician information, and user-customized exercise information.

7. The method of claim 6, characterized in that when providing the user-customized tea information or the user-customized drink information, medicinal herbs of Oriental medicine and components of Western medicine are first shown respectively, and Oriental medicine or Western medicine which comprise the corresponding components is provided when the user makes a selection.

8. The method of claim 1, wherein the user-customized health content information recommendation step comprises extracting only information which matches with the physical constitution analysis-based health content information from among the deep learning-based health content information generated through the deep learning-based health content information generation step to display as the user-customized health content information.

9. The method of claim 1, wherein the user-customized health content information recommendation step comprises:
a user preference analysis step of analyzing a user preference on a treatment remedy;
a user preference based display step of displaying only user-customized health content information corresponding to the analyzed user preference on the treatment remedy from among the deep learning-based health content information, and the physical constitution analysis-based health content information.

10. The method of claim 9, wherein the user preference analysis step comprises receiving a survey response from the user on which treatment method from among a folk remedy and a medical treatment remedy is preferred and identifying the user preference on the treatment remedy based on the input survey response, and the user preference-based display step comprises displaying only the user health content information of any one from among the deep learning-based health content information and the physical constitution analysis-based health content information according to the identified user preference on the treatment remedy.

11. The method of claim 10, wherein the user preference-based display step comprises displaying only the deep learning-based health content information based on the user preference on the treatment remedy being identified as a medical treatment remedy, and displaying only the physical constitution analysis-based health content information based on the user preference on the treatment remedy being identified as a folk remedy.
